(19) United States
(12) Patent Application Publication
(10) Pub. No.: US 2024/0215569 A1
(43) Pub. Date: Jul. 4, 2024

(54) METHODS, COMPOSITIONS, AND KITS FOR THE PRESERVATION OF NUCLEIC ACIDS

(71) Applicant: Gateway Genomics, LLC, San Diego, CA (US)

(72) Inventor: Christopher Jacob, La Jolla, CA (US)

(21) Appl. No.: 18/292,321

(22) PCT Filed: Jul. 27, 2022
(86) PCT No.: PCT/US2022/074222

§ 371 Date: Jan. 25, 2024

Related U.S. Application Data
(60) Provisional application No. 63/225,947, filed on Jul. 27, 2021.

(51) Int. Cl. A01N 1/02

(52) U.S. Cl. CPC A01N 1/0221; A01N 1/0226

(57) ABSTRACT

The disclosure relates to methods, compositions, and kits for the preservation of nucleic acids. The disclosure also provides methods, compositions, and kits for preserving nucleic acids in biological samples (e.g., cell-free DNA in blood or saliva).

### FIELD OF THE INVENTION

The disclosure relates to methods, compositions, and kits for the preservation of nucleic acids. The disclosure also provides methods, compositions, and kits for preserving nucleic acids in biological samples (e.g., cell-free DNA in blood or saliva).

### BACKGROUND OF THE INVENTION

Genetic testing is the analysis of nucleic acids to provide information about a person's genes or chromosomes. Millions of genetic tests are performed each year. Typically, a biological specimen containing nucleic acids is collected from a person and then processed and analyzed at the point of collection or subsequently in a clinical laboratory.

Nucleic acids in biological samples undergo degradation outside of the body. This can occur from nuclease enzyme activity in the biological sample or from exposure to high or low temperatures during transit. Additionally, cells present in biological samples (e.g., blood, saliva) will break down and lyse within a few hours from being removed from the body.

Accordingly, there is a need in the art for methods, compositions, and kits useful for preserving nucleic acids (DNA and RNA) in biological samples. Additionally, there is a need in the art for methods, compositions, and kits for stabilizing cells in biological samples. The disclosure meets this need by providing methods, compositions, and kits for preserving nucleic acids in or obtained from biological samples. The disclosure further provides novel methods, compositions, and kits for stabilizing cells in or obtained from biological samples.

### SUMMARY OF THE INVENTION

The disclosure provides compositions for preserving nucleic acids in a biological sample, the composition comprising one or more agents comprising an anticoagulant agent, a cell stabilizer agent, a cryoprotective agent, and/or a hypertonic agent, or any combination thereof.

In some embodiments, the anticoagulant agent is ethylenediaminetetraacetic acid (EDTA), ethylene glycol-bis(β-aminoethyl ether)-N,N,N′,N′-tetraacetic acid (EGTA), sodium heparin, lithium heparin, and/or sodium citrate. In certain embodiments, the amount of any anticoagulant within the preservative may generally be at least about 0.01% by weight. The amount of any anticoagulant within the preservative may generally be less than about 70% by weight. The amount of any anticoagulant within the preservative may generally be about 0.01%, about 0.1%, about 1%, about 5%, about 10%, about 20%, about 30%, about 40%, about 50%, about 60%, or about 70% by weight.

In certain embodiments, the cell stabilizer agent is an alkali chloride salt, a monosaccharide, a disaccharide, a polysaccharide, an antimicrobial preservative, serum albumin, and/or an amino acid. In other embodiments, the cell stabilizer is phenoxyethanol, sodium benzoate, and/or ethylhexylglycerin. In certain embodiments, the amount of any cell stabilizer within the preservative may generally be at least about 0.01% by weight. The amount of any cell stabilizer within the preservative may generally be less than about 70% by weight. The amount of any cell stabilizer within the preservative may generally be about 0.01%, about 0.1%, about 1%, about 5%, about 10%, about 20%, about 30%, about 40%, about 50%, about 60%, or about 70% by weight.

In some embodiments, the cryoprotective agent is glycerol, ethylene glycol, polyethylene glycol (PEG), propylene glycol, polyvinylpyrrolidone (PVP), carboxylated poly-1-lysine (COOH-PLL), hydroxyethyl starch (HES), dextrose, adenine, d-mannitol, sucrose, trehalose, and/or dimethylsulfoxide (DMSO). In certain embodiments, the amount of any cryoprotectant within the preservative may generally be at least about 0.01% by weight. The amount of any cryoprotectant within the preservative may generally be less than about 70% by weight. The amount of any cryoprotectant within the preservative may generally be about 0.01%, about 0.1%, about 1%, about 5%, about 10%, about 20%, about 30%, about 40%, about 50%, about 60%, or about 70% by weight.

In some embodiments, the hypertonic agent is a salt or a sugar. In some embodiments, the salt is sodium chloride (NaCl). In certain embodiments, the NaCl is in the preservative is about 0.01%, about 0.45%, about 0.9%, about 1.8%, about 2.7%, about 3.6%, about 5%, about 10%, about 20%, about 30%, about 40%, about 50%, about 60%, or about 70% by weight.

In some embodiments, the alkali metal chloride salt is LiCl, NaCl, KCl, RbCl, and/or CsCl. In some embodiments, the amount of any alkali metal chloride salt within the preservative may generally be at least about 0.01% by weight. The amount of any alkali metal chloride salt within the preservative may generally be less than about 70% by weight. The amount of any alkali metal chloride salt within the preservative may generally be about 0.01%, about 0.1%, about 1%, about 5%, about 10%, about 20%, about 30%, about 40%, about 50%, about 60%, or about 70% by weight.

In some embodiments, the monosaccharide is glucose, fructose, and/or galactose. In some embodiments, the amount of any monosaccharide within the preservative may generally be at least about 0.01% by weight. The amount of any monosaccharide within the preservative may generally be less than about 70% by weight. The amount of any monosaccharide within the preservative may generally be about 0.01%, about 0.1%, about 1%, about 5%, about 10%, about 20%, about 30%, about 40%, about 50%, about 60%, or about 70% by weight.

In other embodiments, the disaccharide is sucrose, lactose, maltose, trehalose, chitobiose, and/or cellobiose. In some embodiments, the amount of any disaccharide within the preservative may generally be at least about 0.01% by weight. The amount of any disaccharide within the preservative may generally be less than about 70% by weight. The amount of any disaccharide within the preservative may generally be about 0.01%, about 0.1%, about 1%, about 5%, about 10%, about 20%, about 30%, about 40%, about 50%, about 60%, or about 70% by weight.

In some embodiments, the polysaccharide is cellulose and/or a starch. In certain embodiments, the amount of any polysaccharide within the preservative may generally be at least about 0.01% by weight. The amount of any polysaccharide within the preservative may generally be less than about 70% by weight. The amount of any polysaccharide within the preservative may generally be about 0.01%, about 0.1%, about 1%, about 5%, about 10%, about 20%, about 30%, about 40%, about 50%, about 60%, or about 70% by weight

In some embodiments, the antimicrobial agent is diazolidinyl urea (DU), imidazolidinyl urea (IDU), 1-(3-Chloroallyl)-3,5,7-triaza-1-azoniaadamantane chloride or hexamethylenetetramine chloroallyl chloride (quaternium-15), 2-bromo-2.-nitropropane-1,3-diol, 5-bromo-5-nitro-1,3-dioxane (bronidox), 1,3-Bis(hydroxymethyl)-5,5-dimethylimidazolidine-2,4-dione (DMDM hydantoin), sodium hydroxymethylglycinate, (Benzyloxy)methanol (benzylhemiformal), methenamine, and/or polyoxymethylene urea (polynoxylin). In certain embodiments, the amount of any polysaccharide within the antimicrobial preservative may generally be at least about 0.01% by weight. The amount of any polysaccharide within the antimicrobial preservative may generally be less than about 70% by weight. The amount of any polysaccharide within the antimicrobial preservative may generally be about 0.01%, about 0.1%, about 1%, about 5%, about 10%, about 20%, about 30%, about 40%, about 50%, about 60%, or about 70% by weight.

In some embodiments, the amino acid is glycine, alanine, beta alanine, valine, leucine, isoleucine, methionine, phenylalanine, tryptophan, proline, serine, threonine, cysteine, tyrosine, asparagine, glutamine, aspartic acid, glutamic acid, lysine, arginine, histidine, creatine, n-acetyl cysteine, carnitine, taurine, citrulline, citrulline malate, and/or theanine. In certain embodiments, the amount of any amino acid within the preservative may generally be at least about 0.01% by weight. The amount of any amino acid within the preservative may generally be less than about 70% by weight. The amount of any amino acid within the preservative may generally be about 0.01%, about 0.1%, about 1%, about 5%, about 10%, about 20%, about 30%, about 40%, about 50%, about 60%, or about 70% by weight

In other embodiments, the composition further comprises a solution. In some embodiments, the solution is water, phosphate-buffered saline (PBS), sodium dodecyl sulfate (SDS), 4-(2-hydroxyethyl)-1-piperazineethanesulfonic acid buffer (HEPES), piperazine-N,N′-bis(2-ethanesulfonic acid) buffer (PIPES), Ficoll, Tris-EDTA (TE), Tris-HCl, TAE buffer, and/or formamide.

In some embodiments, the biological sample is selected from the group consisting of whole blood, serum, plasma, urine, interstitial fluid, peritoneal fluid, tears, saliva, cerebrospinal fluid, cell and/or bacterial culture supernatants, cervical swab, buccal swab, tissues, organs, and environmental materials.

In some embodiments, the biological sample of the disclosure comprises blood. In some embodiments, about 0.1-10 mL of blood is obtained from a subject. In other embodiments, about 10-50 mL of blood is obtained from a subject. Blood can be obtained from any suitable area of the body, including an arm, a leg, a finger, or blood accessible through a central venous catheter. In some embodiments, blood is collected from the finger using a lancet. In other embodiments, blood is collected from the arm via venipuncture. In yet other embodiments, blood is collected using a TAP blood collection device (Seventh Sense Biosystems, MA). In some embodiments, blood is collected following a treatment or activity. For example, blood can be collected following a medical exam. The timing of collection can also be coordinated to increase the amount of cell-free fetal nucleic acids present in the sample. For example, blood can be collected following exercise.

The present disclosure provides compositions for preserving nucleic acids. In some embodiments, the nucleic acid is DNA or RNA, or a combination thereof. In certain embodiments, the DNA is cell-free DNA. In other embodiments, the cell-free DNA is cell-free fetal DNA (cffDNA) or cell-free tumor DNA (cftDNA). In yet other embodiments, the RNA is messenger RNA (mRNA) or non-coding RNA (ncRNA).

In some embodiments, the biological sample is obtained from a pregnant subject.

In still other embodiments, the biological sample is obtained from a subject who has been diagnosed or is suspected of having cancer. In some embodiments the cancer is breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors.

In other embodiments, the biological sample is obtained from a subject for purposes of detecting a genetic mutation (e.g., a BRCA mutation). In other embodiments, the biological sample is obtained from a subject for purposes of ancestry analysis. In still other embodiments, the biological sample is obtained from a subject for purposes of performing a genome-wide association study.

Compositions of the disclosure can be located within or subsequently added to a biological sample collection device. In some embodiments, the device is an evacuated collection container (e.g., a blood tube). In other embodiments, the device is a TAP blood collection device. In yet other embodiments, the device is a microcentrifuge tube. In still other embodiments, the device is a capillary blood collection tube (e.g., a BD microtainer blood collection tube). In certain embodiments, the device is a saliva collection tube (e.g., an Oragene saliva collection tube). In yet other embodiments, the device is a urine collection container. In some embodiments, a preservative composition of the disclosure is located in the collection device prior to sample collection. In other embodiments, a preservative composition of the disclosure is added to the collection device after the sample has been collected into the device.

The present disclosure provides methods for preserving nucleic acids in a biological sample. In some embodiments, the disclosure provides a method comprising: contacting a biological sample comprising nucleic acids with a preservative, wherein the preservative comprises one or more anticoagulant agent, cell stabilizer agent, cryoprotective agent, hypertonic agent, or any combination thereof. In some embodiments, the biological sample is contacted with the preservative in a collection device. In other embodiments, the nucleic acids are DNA or RNA, or a combination thereof. In certain embodiments, the DNA is cell-free DNA. In other embodiments, the cell-free DNA is cell-free fetal DNA (cffDNA) or cell-free tumor DNA (cftDNA). In yet other embodiments, the RNA is messenger RNA (mRNA) or non-coding RNA (ncRNA). In some embodiments, the biological sample is selected from the group consisting of whole blood, serum, plasma, urine, interstitial fluid, peritoneal fluid, tears, saliva, cerebrospinal fluid, cell and/or bacterial culture supernatants, cervical swab, buccal swab, tissues, organs, and environmental materials.

In other embodiments, the disclosure provides kits comprising: a sample collection device and a composition comprising one or more anticoagulant agents, one or more cell stabilizer agents, one or more cryoprotective agents, one or more hypertonic agents, or any combination thereof. In some embodiments, the composition is contained inside the sample collection device. In other embodiments, the composition in the kit is not contained in the sample collection device.

In some embodiments, the biological sample is incubated with a preservative of the disclosure and can be processed at any time after being collected from the subject. In some embodiments, the biological sample is processed within 1 hour, within 24 hours, or within 48 hours after coming into contact with a preservative of the disclosure. In other embodiments, the biological sample is not processed for at least 6 hours, at least 8 hours, at least 10 hours, at least 12 hours, at least 1 day, at least 2 days, at least 3 days, at least 1 week, at least, 2 weeks, or at least 4 weeks after being contacted with a preservative of the disclosure. In some embodiments, the nucleic acids in the biological sample are detected within 1 hour, within 2 hours, within 4 hours, within 24 hours, or within 48 hours after coming into contact with a preservative of the disclosure. In other embodiments, the nucleic acids in the biological sample are not isolated and/or detected for at least 6 hours, at least 8 hours, at least 10 hours, at least 12 hours, at least 1 day, at least 2 days, at least 3 days, at least 1 week, at least, 2 weeks, or at least 4 weeks after coming into contact with a preservative of the disclosure.

These and other embodiments of the present disclosure will readily occur to those of skill in the art in light of the disclosure herein, and all such embodiments are specifically contemplated.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

### DESCRIPTION OF THE INVENTION

It is to be understood that the disclosure is not limited to the particular methodologies, protocols, cell lines, assays, and reagents described herein, as these may vary. It is also to be understood that the terminology used herein is intended to describe particular embodiments of the present disclosure, and is in no way intended to limit the scope of the present disclosure as set forth in the appended claims.

It must be noted that as used herein and in the appended claims, the singular forms “a,” “an,” and “the” include plural references unless context clearly dictates otherwise. Thus, for example, a reference to “a nucleic acid” includes a plurality of such nucleic acids, a reference to a “composition” is a reference to one or more compositions and to equivalents thereof known to those skilled in the art, and so forth.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All publications cited herein are incorporated herein by reference in their entirety for the purpose of describing and disclosing the methodologies, reagents, and tools reported in the publications that might be used in connection with the disclosure. Nothing herein is to be construed as an admission that the disclosure is not entitled to antedate such disclosure by virtue of prior invention.

The practice of the present disclosure will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The disclosure relates, in part, to the discovery of compositions useful for preserving nucleic acids in biological samples.

The disclosure is based, in part, on the discovery of unexpected improvements in the preservation and stabilization of cell-free nucleic acids and cells in biological samples. The disclosure demonstrates that nucleic acids and cells present in biological samples may be preserved and stabilized for an extended period of time to prevent degradation of the nucleic acids and cells in the sample and permit subsequent genetic testing with the nucleic acids obtained from the biological sample.

The disclosure provides methods for preserving nucleic acids in a biological sample using a preservative composition of the disclosure. In some embodiments, the methods preserve nucleic acids in a biological sample for at least 4 hours, 8 hours, 12 hours, 1 day, 2 days, 3 days, 7 days, 14 days, and/or 28 days. The disclosure also provides methods for stabilizing cells in a biological sample using a preservative composition of the disclosure. In some embodiments, the methods stabilize cells in a biological sample for at least 4 hours, 8 hours, 12 hours, 1 day, 2 days, 3 days, 7 days, 14 days, and/or 28 days.

The disclosure also provides preservative compositions for use in the methods described herein. Such compositions may include one or more anticoagulant agents, one or more cell stabilizer agents, one or more cryoprotective agents, one or more hypertonic agents, or any combination thereof.

The present disclosure further provides kits for preserving nucleic acids in biological samples collected from a subject. In these embodiments, the kits comprise a sample collection device and a preservative composition of the disclosure.

The section headings are used herein for organizational purposes only, and are not to be construed as in any way limiting the subject matter described herein.

### Biological Sample

The disclosure provides methods, compositions, and kits for preservation of nucleic acids (e.g., DNA, RNA). Generally, the methods of the disclosure involve the stabilization of cells and preservation of nucleic acids in a biological sample obtained from a subject. A biological sample comprising nucleic acids may be obtained from a subject. The biological sample obtained from the subject is typically blood, but can be any sample from bodily fluids, tissue or cells comprising the nucleic acids to be analyzed. The biological sample may include, but is not limited to, whole blood, serum, plasma, urine, a cervical swab, saliva, a buccal swab, and/or amniotic fluid.

In some embodiments, the biological sample of the disclosure can be obtained from blood. In some embodiments, about 0.1-10 mL of blood is obtained from a subject. In other embodiments, about 10-50 mL of blood is obtained from a subject. Blood can be obtained from any suitable area of the body, including an arm, a leg, a finger, or blood accessible through a central venous catheter. In some embodiments, blood is collected from the finger using a lancet. In other embodiments, blood is collected from the arm via venipuncture. In yet other embodiments, blood is collected from the arm using a TAP device (Seventh Sense Biosystems, MA). In some embodiments, blood is collected following a treatment or activity. For example, blood can be collected following a medical exam. The timing of collection can also be coordinated to increase the amount of nucleic acids present in the sample. For example, blood can be collected following exercise.

The volume of the biological sample obtained from the subject may be 10 ul to 20 ml. In some embodiments, the volume of the sample used to detect nucleic acids is a microvolume. In certain embodiments, the microvolume is about 1,000 ul, about 900 ul, about 800 ul, about 700 ul, about 600 ul, about 500 ul, about 400 ul, about 300 ul, about 200 ul, about 150 ul, about 100 ul, about 50 ul, about 25 ul, about 10 ul. Blood samples are typically processed within a few hours from the time of collection to prevent significant degradation of the nucleic acids by enzymes present in blood. The methods of the disclosure enable the biological sample to be processed up to several days or months after being collected from the subject. In some embodiments, the biological sample is processed within 1 hour, within 24 hours, or within 48 hours. In other embodiments, the biological sample is not processed for at least 6 hours, at least 8 hours, at least 10 hours, at least 12 hours, at least 1 day, at least 2 days, at least 3 days, at least 1 week, at least, 2 weeks, or at least 4 weeks.

The biological sample should be free of contaminating DNA from a source different than the subject (e.g., touch DNA from another person). In some aspects, maternal blood is collected from a site on the body which is generally free of contaminating Y-chromosome DNA. In some embodiments, the site of blood collection is the upper arm. A TAP blood specimen collection device is used to collect a blood sample. The TAP Blood Collection Device is a single-use, sterilized blood collection and transportation device that uses a combination of two mechanisms, capillary action and vacuum extraction. The device consists of an integrated reservoir with a visual fill indicator window. The device is designed to collect and contain approximately 100-500 μL of capillary whole blood. The internal fluid path is coated with lithium heparin, EDTA, EGTA, or other agents and/or compositions of the disclosure. The top of the device includes a green button and a fill indicator window. The base of the device includes a release liner that covers a layer of hydrogel adhesive. The hydrogel adhesive seals to the skin and holds the device in place during use. The TAP device contains an array of microneedles in order to puncture through the skin. The microneedles are activated by a spring, released by pushing a green button on the device. The device is provided sterile in a tray and foil pouch. A preservative or cell stabilizer can optionally be used in the TAP device to allow for DNA analysis more than 6 hours after a blood sample is collected. In some embodiments, the blood sample is processed 6 hours, 8 hours, 10 hours, 12 hour, 24 hours, 48 hour, 72 hours, 96 hours, 120 hours, 144 hours, or 168 hours after collection. In some embodiments, the blood is stabilized in the TAP device with a preservative such that DNA concentration in the plasma portion of the blood sample remain relatively constant for up to 7 to 14 days post collection. In some embodiment, the preservative in the TAP device prevents significant genomic DNA contamination in blood samples for up to 7 to 14 days post collection

In some embodiments, the TAP device including the preservative, facilitates storage of the blood sample collected in the tube at room temperature for at least, or about 14 days without cell lysis and without cell-free nucleic acid degradation of the blood sample due to DNase and RNase activity after blood draw.

### Subjects

The disclosure provides methods, compositions, and kits for the preservation of nucleic acids in a biological sample collected from a subject. The disclosure is applicable to a variety of different organisms, including for example, vertebrates, large animals and primates. In one embodiment, the subject is a mammalian subject, and in other embodiments, the subject is a human subject. However, although applications with humans are clearly foreseen, veterinary applications are also envisaged here. In some embodiments, the subject is a human, a monkey, a dog, a pig, a bovine, a rabbit, a guinea pig, and/or a rodent.

The methods, compositions, and kits of the disclosure are useful for preserving nucleic acids and stabilizing cells in biological samples from subjects with diseases or health conditions. In some embodiments, the subject has cancer. In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

In some embodiments, the present disclosure enables a medical practitioner to diagnose or prognose one or more cancers in a subject. In other embodiments, the present disclosure enables a medical practitioner to rule out or eliminate one or more cancers as a diagnostic possibility. In other embodiments, the methods of the present disclosure allow a medical practitioner to identify the origin of a cancer. In yet other embodiments, the present disclosure enables a medical practitioner to identify a subject at risk of developing cancer. In other embodiments, the present disclosure enables a medical practitioner to predict whether a subject will later develop cancer. In further embodiments, the present disclosure enables a medical practitioner to prescribe a therapeutic regimen or predict benefit from therapy in a subject having cancer.

In other embodiments, the subject has a neurological disease or disorder. In some embodiments, neurological disease or disorder is Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia and/or Parkinson's disease.

In some embodiments, the subject is a pregnant subject. The pregnancy may be the result of natural conception (i.e., a natural pregnancy) of result from use of assisted reproductive technology (e.g., in-vitro fertilization). In some embodiments, the pregnant subject has used assisted reproductive technology (ART) to become pregnant. In some aspects, the assisted reproductive technology is in-vitro fertilization, use of fertility medication (e.g., clomifene), ovulation induction, cryopreservation, and/or intracytoplasmic sperm injection. In some embodiments, the pregnant subject has a high-risk pregnancy. In other embodiments, the pregnant subject is a carrier of a sex-linked recessive disease or disorder.

In some embodiments, the present disclosure enables a medical practitioner to diagnose or prognose one or more neurological disorders in a subject. In other embodiments, the present disclosure enables a medical practitioner to rule out or eliminate one or more neurological diseases as a diagnostic possibility. In other embodiments, the methods of the present disclosure allow a medical practitioner to distinguish some forms of FTD from Alzheimer's disease. In yet other embodiments, the present disclosure enables a medical practitioner to identify a subject at risk of developing a neurological disorder. In other embodiments, the present disclosure enables a medical practitioner to predict whether a subject will later develop a neurological disorder. In further embodiments, the present disclosure enables a medical practitioner to prescribe a therapeutic regimen or predict benefit from therapy in a subject having a neurological disorder.

The disclosure provides methods, compositions, and kits useful for preserving nucleic acids and stabilizing cells in biological samples from pregnant subjects at various timepoints in pregnancy. Gestational age is a measure of the age of a pregnancy which is taken from the beginning of the woman's last menstrual period (LMP), or the corresponding age of the gestation as estimated by a more accurate method if available. Such methods include adding 14 days to a known duration since fertilization (as is possible in in vitro fertilization), or by obstetric ultrasonography. In some embodiments, the gestational age of the fetus is 2 weeks, 3 weeks, 4 weeks, 5 weeks, 6 weeks, 7 weeks, 8 weeks, 9 weeks, 10 weeks, 11 weeks, or 12 weeks. In some embodiments, the gestational age of the fetus is 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days 42 days, 49 days, 56 days, 63 days, 70 days, 77 days, or 84 days.

The present disclosure provides methods for diagnosing or prognosing placental disease in a subject, identifying a subject at risk of developing a placental disease, or prescribing a therapeutic regimen or predicting benefit from therapy in a subject having a placental disease. Generally, a placental disease is any disease, disorder, or pathology of the placenta. The methods and biomarkers of the present disclosure may also be used for fetal assessment or diagnosis of fetal disorders, such as, for example, fetal alcohol syndrome or fetal genetic abnormalities.

The present disclosure provides methods for diagnosing or prognosing an immunological disorder in a subject, identifying a subject at risk of developing an immunological disorder, or prescribing a therapeutic regimen or predicting benefit from therapy in a subject having an immunological disorder. Immunological disorders are diseases or conditions caused by a dysfunction of the immune system and include allergy, asthma, autoimmune diseases, autoinflammatory syndromes and immunological deficiency syndromes.

### Compositions and Agents

The disclosure provides compositions for preserving nucleic acids in a biological sample, the composition comprising one or more agents comprising an anticoagulant agent, cell stabilizer agent, cryoprotective agent, and/or hypertonic agent, or any combination thereof.

In some embodiments, the anticoagulant agent is ethylenediaminetetraacetic acid (EDTA), ethylene glycol-bis(β-aminoethyl ether)-N,N,N′,N′-tetraacetic acid (EGTA), sodium heparin, lithium heparin, and/or sodium citrate. In certain embodiments, the amount of any anticoagulant within the preservative may generally be at least about 0.01% by weight. The amount of any anticoagulant within the preservative may generally be less than about 70% by weight. The amount of any anticoagulant within the preservative may generally be about 0.01%, about 0.1%, about 1%, about 5%, about 10%, about 20%, about 30%, about 40%, about 50%, about 60%, or about 70% by weight.

In certain embodiments, the cell stabilizer agent is an alkali chloride salt, a monosaccharide, a disaccharide, a polysaccharide, an antimicrobial preservative, serum albumin, and/or an amino acid. In certain embodiments, the amount of any cell stabilizer within the preservative may generally be at least about 0.01% by weight. The amount of any cell stabilizer within the preservative may generally be less than about 70% by weight. The amount of any cell stabilizer within the preservative may generally be about 0.01%, about 0.1%, about 1%, about 5%, about 10%, about 20%, about 30%, about 40%, about 50%, about 60%, or about 70% by weight.

Freezing of biological samples is generally accompanied by numerous undesirable outcomes such as intra- and extracellular freezing damage and osmotic stress. To prevent these adverse effects, cryoprotective agents can be used in the preservative compositions of the disclosure. In some embodiments, the cryoprotective agent is glycerol, ethylene glycol, polyethylene glycol (PEG), propylene glycol, polyvinylpyrrolidone (PVP), carboxylated poly-1-lysine (COOH-PLL), hydroxyethyl starch (HES), dextrose, adenine, d-mannitol, sucrose, trehalose, and/or dimethylsulfoxide (DMSO). In certain embodiments, the amount of any cryoprotectant within the preservative may generally be at least about 0.01% by weight. The amount of any cryoprotectant within the preservative may generally be less than about 70% by weight. The amount of any cryoprotectant within the preservative may generally be about 0.01%, about 0.1%, about 1%, about 5%, about 10%, about 20%, about 30%, about 40%, about 50%, about 60%, or about 70% by weight.

A hypertonic solution is a particular type of solution that has a greater concentration of solutes on the outside of a cell when compared with the inside of a cell. In some embodiments, the preservative composition comprises a hypertonic agent. In some embodiments, the hypertonic agent is a salt or a sugar. In some embodiments, the salt is sodium chloride (NaCl). In certain embodiments, the NaCl is in the preservative is about 0.01%, about 0.45%, about 0.9%, about 1.8%, about 2.7%, about 3.6%, about 5%, about 10%, about 20%, about 30%, about 40%, about 50%, about 60%, or about 70% by weight.

Alkali metal chloride salts can be used in the preservative compositions of the disclosure. In some embodiments, the alkali metal chloride salt is LiCl, NaCl, KCl, RbCl, and/or CsCl. In some embodiments, the amount of any alkali metal chloride salt within the preservative may generally be at least about 0.01% by weight. The amount of any alkali metal chloride salt within the preservative may generally be less than about 70% by weight. The amount of any alkali metal chloride salt within the preservative may generally be about 0.01%, about 0.1%, about 1%, about 5%, about 10%, about 20%, about 30%, about 40%, about 50%, about 60%, or about 70% by weight.

Addition of sugars to a preservative composition of the disclosure can help regulate the osmotic pressure of diluents. This is done by inducing cell dehydration and avoiding ice crystal formation. Monosaccharides, disaccharides, and polysaccharides can be used for this purpose. In some embodiments, the monosaccharide is glucose, fructose, and/or galactose. In some embodiments, the amount of any monosaccharide within the preservative may generally be at least about 0.01% by weight. The amount of any monosaccharide within the preservative may generally be less than about 70% by weight. The amount of any monosaccharide within the preservative may generally be about 0.01%, about 0.1%, about 1%, about 5%, about 10%, about 20%, about 30%, about 40%, about 50%, about 60%, or about 70% by weight.

In other embodiments, the disaccharide is sucrose, lactose, maltose, trehalose, chitobiose, and/or cellobiose. In some embodiments, the amount of any disaccharide within the preservative may generally be at least about 0.01% by weight. The amount of any disaccharide within the preservative may generally be less than about 70% by weight. The amount of any disaccharide within the preservative may generally be about 0.01%, about 0.1%, about 1%, about 5%, about 10%, about 20%, about 30%, about 40%, about 50%, about 60%, or about 70% by weight.

In some embodiments, the polysaccharide is cellulose and/or a starch. In certain embodiments, the amount of any polysaccharide within the preservative may generally be at least about 0.01% by weight. The amount of any polysaccharide within the preservative may generally be less than about 70% by weight. The amount of any polysaccharide within the preservative may generally be about 0.01%, about 0.1%, about 1%, about 5%, about 10%, about 20%, about 30%, about 40%, about 50%, about 60%, or about 70% by weight

In some embodiments, the antimicrobial agent is diazolidinyl urea (DU), imidazolidinyl urea (IDU), quaternium-15, 5-bromo-5-nitro-1,3-dioxane, DMDM hydantoinin, sodium hydroxymethylglycinate, benzylhemiformal, bronopol, and/or bronidox. In certain embodiments, the amount of any polysaccharide within the antimicrobial preservative may generally be at least about 0.01% by weight. The amount of any polysaccharide within the antimicrobial preservative may generally be less than about 70% by weight. The amount of any polysaccharide within the antimicrobial preservative may generally be about 0.01%, about 0.1%, about 1%, about 5%, about 10%, about 20%, about 30%, about 40%, about 50%, about 60%, or about 70% by weight.

Amino acids can be used in the compositions of the disclosure to help stabilize cell membranes. In some embodiments, the amino acid is glycine, alanine, beta alanine, valine, leucine, isoleucine, methionine, phenylalanine, tryptophan, proline, serine, threonine, cysteine, tyrosine, asparagine, glutamine, aspartic acid, glutamic acid, lysine, arginine, histidine, creatine, n-acetyl cysteine, carnitine, taurine, citrulline, citrulline malate, and/or theanine. In certain embodiments, the amount of any amino acid within the preservative may generally be at least about 0.01% by weight. The amount of any amino acid within the preservative may generally be less than about 70% by weight. The amount of any amino acid within the preservative may generally be about 0.01%, about 0.1%, about 1%, about 5%, about 10%, about 20%, about 30%, about 40%, about 50%, about 60%, or about 70% by weight

The agents used in the preservative compositions of the disclosure can be dissolved in a solution. Such solutions can then be added to a sample collection device such as a blood tube. In some embodiments, the composition of the disclosure further comprises a solution. In some embodiments, the solution is water, phosphate-buffered saline (PBS), sodium dodecyl sulfate (SDS), 4-(2-hydroxyethyl)-1-piperazineethanesulfonic acid buffer (HEPES), piperazine-N,N′-bis(2-ethanesulfonic acid) buffer (PIPES), Ficoll, Tris-EDTA (TE), Tris-HCl, TAE buffer, and/or formamide.

### Nucleic Acids

The present disclosure provides compositions for preserving nucleic acids. In some embodiments, the nucleic acid is DNA or RNA, or a combination thereof. In certain embodiments, the DNA is cell-free DNA. In other embodiments, the cell-free DNA is cell-free fetal DNA (cffDNA) or cell-free tumor DNA (cftDNA). The nucleic acids can be genomic DNA or mitochondrial DNA. In some embodiments, the nucleic acids can be cellular DNA. In yet other embodiments, the RNA is messenger RNA (mRNA) or non-coding RNA (ncRNA). The nucleic acids may be isolated from whole blood, plasma, and/or serum.

### Devices

Compositions of the disclosure can be located within or subsequently added to a biological sample collection device. In some embodiments, the device is an evacuated collection container (e.g., a blood tube). In other embodiments, the device is a TAP blood collection device. In yet other embodiments, the device is a microcentrifuge tube. In still other embodiments, the device is a capillary blood collection tube (e.g., a BD microtainer blood collection tube). In certain embodiments, the device is a saliva collection tube (e.g., an Oragene saliva collection tube). In yet other embodiments, the device is a urine collection container. In some embodiments, a preservative composition of the disclosure is located in the collection device prior to sample collection. In other embodiments, a preservative composition of the disclosure is added to the collection device after the sample has been collected into the device.

In some embodiments, a TAP blood specimen collection device is used to collect a blood sample. The TAP Blood Collection Device is a single-use, sterilized blood collection and transportation device that uses a combination of two mechanisms, capillary action and vacuum extraction. The device consists of an integrated reservoir with a visual fill indicator window. The device is designed to collect and contain approximately 100-500 μL of capillary whole blood. The internal fluid path is coated with lithium heparin, EDTA, EGTA, or other anticoagulants and/or preservatives. The top of the device includes a green button and a fill indicator window. The base of the device includes a release liner that covers a layer of hydrogel adhesive. The hydrogel adhesive seals to the skin and holds the device in place during use. The TAP device contains an array of microneedles in order to puncture through the skin. The microneedles are activated by a spring, released by pushing a green button on the device. The device is provided sterile in a tray and foil pouch. A preservative or cell stabilizer can optionally be used in the TAP device to allow for DNA analysis more than 6 hours after a blood sample is collected. In some embodiments, the blood sample is processed 6 hours, 8 hours, 10 hours, 12 hour, 24 hours, 48 hour, 72 hours, 96 hours, 120 hours, 144 hours, or 168 hours after collection. In some embodiments, the blood is stabilized in the TAP device with a preservative such that DNA concentration in the plasma portion of the blood sample remain relatively constant for up to 7 to 14 days post collection. In some embodiment, the preservative in the TAP device prevents significant genomic DNA contamination in blood samples for up to 7 to 14 days post collection.

In some embodiments, a TAP device comprising a composition of the disclosure, facilitates storage of the blood sample collected in the tube at room temperature for at least, or about 14 days without cell lysis and without cell-free nucleic acid degradation of the blood sample due to DNase and RNase activity after blood draw.

In other embodiments, a composition of the disclosure is embedded in a membrane or matrix and a biological sample is brought into contact with the membrane or matrix to preserve the nucleic acids in the biological sample.

### Methods

As disclosed herein, contacting a blood or plasma sample with a composition of the disclosure allows a biological sample to be stored for a period of time prior to isolating and/or testing the nucleic acids. A blood or plasma sample may be drawn at one location (e.g., a health care facility), contacted with the composition, and later transported to a different remote location (e.g., a laboratory, such as one that is separately housed at a distance of at least about 1 kin, 2 km, 3 km, or further away from the draw site) for the nucleic acid isolation and/or testing process. The nucleic acids can be isolated from the blood or plasma sample and tested at the remote location and the resulting diagnostic information may be reported to the site of the original blood draw. The nucleic acid isolation process may be performed at one remote location and the resulting data can be analyzed to identify the presence, absence or relative severity of a disease state at a third location. Alternatively, the results of the nucleic acid isolation process may be sent back to the site of the initial blood draw and analyzed there. The resulting diagnostic information may then be sent to a third location or back to the remote location or the site of the initial blood draw. The nucleic acids can be DNA or RNA. The DNA can be genomic DNA (gDNA) or cell-free DNA (cfDNA).

At any time after the initial contact of the biological sample (e.g., blood or plasma) with a composition of the disclosure, the biological sample can be treated to isolate the cell-free nucleic acids located within the sample, such as for example, cfDNA in plasma. The nucleic acids may be isolated using any isolation method including those methods disclosed in U.S. Patent Publication No. 2009/0081678, incorporated by reference herein. The protective agent may aid in maintaining the integrity of blood cell membranes (e.g., the cell membranes remain intact), so that nucleic acids are not released into the sample from blood cells having ruptured cell membranes. Any cell membrane rupture may cause cellular nucleic acids (e.g., genomic DNA) to enter the plasma making isolation of the cell-free nucleic acids (e.g., identifying and separating cell-free nucleic acids from nucleic acids that originated within a blood cell) more difficult. A composition of the disclosure may act to prevent cell lysis so that the blood cells remain intact and substantially all cellular nucleic acids remain intra-cellular to avoid unwanted contamination of the cell-free nucleic acids (e.g., genomic DNA contamination).

The disclosure provides methods for isolating nucleic acids from a biological sample; and analyzing (e.g., by quantity, quality, or both) the isolated nucleic acids for the presence, absence, or severity of a disease or condition. The agent and compositions of the disclosure may be present in an amount and for a time sufficient so that nucleic acids are preserved in the biological sample for at least two hours, at least four hours, at least 12 hours, at least 24 hours, at least 72 hours, and/or at least 30 days. The agents and compositions of the disclosure may be present in an amount so that cells in the biological sample are fixed or stabilized to substantially prevent leaking of cellular nucleic acids outside of the cell (e.g., prevent cellular nucleic acids in blood cells from leaking into plasma). The agents and compositions of the disclosure may be present in an amount so that any cellular nucleic acids that are within cells at the time the biological sample is collected are substantially stabilized against degradation from nucleases in the biological sample.

### Kits

Another aspect of the disclosure encompasses kits for collecting biological samples from subjects or for isolating and/or testing nucleic acids in a biological sample from a subject. A variety of kits having different components are contemplated by the disclosure. Generally speaking, the kit will include the means for obtaining a biological sample from a subject. In another embodiment, the kit will include means for collecting and/or storing a biological sample and instructions for use of the kit contents. In certain embodiments, the kit comprises a means for enriching or isolating nucleic acids in a biological sample. In further aspects, the means for enriching or isolating nucleic acids comprises reagents necessary to enrich or isolate nucleic acids from a biological sample.

The kits of the disclosure may include instructions for decontaminating the site on the subject where the sample will be collected. In certain embodiments, the decontamination is performed by applying bleach to the site of collection, by applying an alcohol wipe to the site of collection, by treating the site of collection with ultra-violet light, by applying chlorhexidine gluconate, hydrogen peroxide, and/or iodine to the site of collection, by applying a brush (e.g., a nail brush) to the site of the collection.

The present disclosure further provides kits for obtaining a biological sample from a subject. The kits may comprise a blood collection tube, a lancet or a device useful for obtaining venous or capillary blood from the subject, a tourniquet, a bandage, an alcohol swab, a nail or skin brush, and instructions for using the kits. In some embodiments, the kits further comprise a decontaminating agent. In certain embodiments, the decontaminating agent is bleach, an alcohol wipe, chlorhexidine gluconate, hydrogen peroxide, and/or iodine. In other embodiments, the device for obtaining venous or capillary blood is a lancet (e.g., BD Microtainer contact-activated lancet), a syringe, and/or a push-button blood collection device (e.g., a TAP device). In some embodiments, the biological sample is collected into a tube, onto a card, and/or a swab.

These and other embodiments of the present disclosure will readily occur to those of ordinary skill in the art in view of the disclosure herein.

### EXAMPLES

The disclosure will be further understood by reference to the following examples, which are intended to be purely exemplary of the disclosure. These examples are provided solely to illustrate the claimed disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description. Such modifications are intended to fall within the scope of the appended claims.

### Example 1: Preservation and Detection of Nucleic Acids in Human Blood Samples

Preservation and detection of nucleic acids in biological samples using a composition of the disclosure was assessed as follows. Two whole blood samples were collected into blood tubes from a human subject. One blood tube contained only EDTA. The other blood tube contained a preservative composition of the disclosure containing an anticoagulant agent and two cell stabilizer agents. Both blood tubes were stored at room temperature and 500 ul aliquots of blood from each tube were tested 3 days after collection. The 500 μL aliquots of blood were centrifuged at 1,600 g for 15 minutes to separate plasma from whole blood. Next, 100 μL of the plasma was taken from the samples and cell-free DNA (cfDNA) was isolated using a cell-free DNA Isolation Kit (ThermoFisher) according to the manufacturer's instructions.

Real-time quantitative polymerase chain reaction (RT-qPCR) was utilized to measure levels of cell-free DNA as follows. Isolated cell-free DNA (5 ul) was dispensed into 96-well plates and reacted with a custom master mix for a final PCR reaction volume of 25 ul per well. An autosomal control gene target was detected to quantify the amount of total cfDNA that was isolated from each sample.

Total DNA levels in the plasma from the EDTA only tube was significantly greater than the DNA levels detected in the plasma from the preservative tube indicating that the cells in the EDTA tube were not stabilized and had released genomic DNA into the plasma. The levels of DNA detected in the sample with the preservative composition were equivalent to DNA levels in plasma obtained from plasma proceeded immediately following collection (i.e., at time zero). These results indicated that preservative compositions of the disclosure are useful for preserving cell-free DNA in whole blood. These results further showed that preservative compositions of the disclosure are useful for stabilizing cells in whole blood. These results suggested that preservative compositions of the disclosure would be useful for preserving nucleic acids in biological samples and for stabilizing cells in biological samples. These results further suggested that the methods, compositions, and kits of the disclosure would be useful for preserving nucleic acids in biological samples.

### Example 2: Blood Cell Stabilization and Nucleic Acid Preservation in Human Blood Samples for 28 Days

Blood cell stabilization and cell-free nucleic acid preservation and detection in human blood samples using a composition of the disclosure was assessed as follows. Venous whole blood was collected into four blood tubes from a human subject. Two blood tubes contained only EDTA (EDTA blood tubes). The other two blood tubes contained a preservative composition of the disclosure (preservative blood tubes) containing an anticoagulant agent and two cell stabilizer agents. Five aliquots (250 ul) of whole blood was obtained from one of the EDTA blood tubes and one of the preservative blood tubes. One of the EDTA blood tubes and one of the preservative blood tubes were centrifuged at 1,600 g for 15 minutes to isolate plasma from the whole blood. Five aliquots of plasma (110 ul) were obtained from those blood tubes. All aliquots, whole blood and plasma, were stored at room temperature for the duration of the experiment. Cell-free DNA (cfDNA) levels were determined in the whole blood and plasma aliquots at day 0, 3, 7, 14, and 28.

Whole blood aliquots were centrifuged as described above to isolate plasma from those aliquots at each timepoint that cfDNA levels were assessed. CfDNA was isolated from plasma samples using a commercial cfDNA isolation kit according to the manufacturer's instructions. Real-time qPCR was used to detect a single copy autosomal gene (total cfDNA) under the following conditions 10 min at 95° C. to allow for the initial denaturation of DNA and polymerase activation, followed by 45 cycles of one minute at 60° C. and 15 seconds at 95° C.

Cycle threshold (CT) values, which represent total cfDNA levels, for the whole blood samples at each timepoint are shown below in Table 1. Total DNA levels in the whole blood samples can be used to determine cell stability. If cells are effectively stabilized in the sample, then cell-free DNA levels will not increase. If the cells are not stabilized then the cells will lyse and release genomic DNA into the plasma. The result is a significant increase in cell-free DNA levels in the blood sample. As shown in Table 1 below, CT values in the EDTA only blood tubes were significantly decreased from baseline at all timepoints after day 0 indicating that cells in the EDTA blood tubes were not stabilized. CT values in the preservative tubes were not significantly changed at any time point indicating that the preservative was effective for stabilizing the cells in whole blood.

TABLE 1
CT Values for Whole Blood Samples,
EDTA and Preservative Blood Tubes

| | CT Value | CT Value |
|---|---|---|
| Time Point | (EDTA Blood | (Preservative Blood |
| (day) | Tube) | Tube) |
| 0 | 32.00 | 33.07 |
| 3 | 29.09 | 33.89 |
| 7 | 24.74 | 33.97 |
| 14 | 20.59 | 32.95 |
| 28 | Undetermined | 30.97 |

Cycle threshold (CT) values, which represent total cfDNA levels, for the plasma samples at each timepoint are shown below in Table 2. Total DNA levels in the plasma samples can be used to determine cell-free DNA preservation. If the cell-free nucleic acids are effectively preserved in the sample, then cell-free DNA levels will not decrease over time. If the cell-free nucleic acids are not preserved then the nucleic acids will be degraded. The result is a significant decrease in cell-free DNA levels in the plasma sample. As shown in Table 2 below, CT values in the EDTA only blood tubes were decreased from baseline at all timepoints after day 0 indicating that cell-free DNA in the EDTA blood tubes were not preserved. Conversely, CT values in the preservative tubes were not significantly changed at any time point indicating that the preservative was effective for preserving the cell-free DNA in plasma.

CT values for the plasma samples at
each timepoint are shown in Table 2.

| | CT Value | CT Value |
|---|---|---|
| Time Point | (EDTA Blood | (Preservative Blood |
| (day) | Tube) | Tube) |
| 0 | 32.36 | 33.75 |
| 3 | 29.86 | 34.11 |
| 7 | 30.54 | 33.31 |
| 14 | 30.24 | 34.10 |
| 28 | 30.41 | 32.95 |

These results showed that preservative compositions of the disclosure are useful for preserving cell-free DNA in plasma. These results further showed that preservative compositions of the disclosure are useful for stabilizing cells in whole blood. These results suggested that preservative compositions of the disclosure would be useful for preserving nucleic acids in biological samples and for stabilizing cells in biological samples. These results further suggested that the methods, compositions, and kits of the disclosure would be useful for preserving nucleic acids in biological samples

Various modifications of the disclosure, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A composition for preserving nucleic acids in a biological sample, the composition comprising one or more agents comprising an anticoagulant agent, a cell stabilizer agent, a cryoprotective agent, and/or a hypertonic agent.

2. The composition of claim 1, wherein the anticoagulant agent is ethylenediaminetetraacetic acid (EDTA), ethylene glycol-bis(β-aminoethyl ether)-N,N,N′,N′-tetraacetic acid (EGTA), sodium heparin, lithium heparin, and/or sodium citrate.

3. The composition of claim 1, wherein the cell stabilizer agent is an alkali chloride salt, a monosaccharide, a disaccharide, a polysaccharide, an antimicrobial preservative, serum albumin, and/or an amino acid.

4. The composition of claim 1, wherein the cell stabilizer is phenoxyethanol, sodium benzoate, and/or ethylhexylglycerin.

5. The composition of claim 1, wherein the cryoprotective agent is glycerol, ethylene glycol, polyethylene glycol (PEG), propylene glycol, polyvinylpyrrolidone (PVP), carboxylated poly-1-lysine (COOH-PLL), hydroxyethyl starch (HES), dextrose, adenine, d-mannitol, sucrose, trehalose, and/or dimethylsulfoxide (DMSO).

6. The composition of claim 1, wherein the hypertonic agent is a salt or a sugar.

7. The composition of claim 6, wherein the salt is sodium chloride (NaCl).

8. The composition of claim 3, wherein the alkali chloride salt is LiCl, NaCl, KCl, RbCl, and/or CsCl.

9. The composition of claim 3, wherein the monosaccharide is glucose, fructose, and/or galactose.

10. The composition of claim 3, wherein the disaccharide is sucrose, lactose, maltose, trehalose, chitobiose, and/or cellobiose.

11. The composition of claim 3, wherein the polysaccharide is cellulose and/or a starch.

12. The composition of claim 3, wherein the antimicrobial agent is diazolidinyl urea (DU), imidazolidinyl urea (IDU), 1-(3-Chloroallyl)-3,5,7-triaza-1-azoniaadamantane chloride or hexamethylenetetramine chloroallyl chloride (quaternium-15), 2-bromo-2.-nitropropane-1,3-diol, 5-bromo-5-nitro-1,3-dioxane (bronidox), 1,3-Bis(hydroxymethyl)-5,5-dimethylimidazolidine-2,4-dione (DMDM hydantoin), sodium hydroxymethylglycinate, (Benzyloxy)methanol (benzylhemiformal), methenamine, and/or polyoxymethylene urea (polynoxylin).

13. The composition of claim 3, wherein the amino acid is glycine, alanine, beta alanine, valine, leucine, isoleucine, methionine, phenylalanine, tryptophan, proline, serine, threonine, cysteine, tyrosine, asparagine, glutamine, aspartic acid, glutamic acid, lysine, arginine, histidine, creatine, n-acetyl cysteine, carnitine, taurine, citrulline, citrulline malate, and/or theanine.

14. The composition of claim 1, wherein the composition further comprises a solution.

15. The composition of claim 14, wherein the solution is water, phosphate-buffered saline (PBS), sodium dodecyl sulfate (SDS), 4-(2-hydroxyethyl)-1-piperazineethanesulfonic acid buffer (HEPES), piperazine-N,N′-bis(2-ethanesulfonic acid) buffer (PIPES), Ficoll, Tris-EDTA (TE), Tris-HCl, TAE buffer, and/or formamide.

16. The composition of claim 1, wherein the biological sample is selected from the group consisting of whole blood, serum, plasma, urine, interstitial fluid, peritoneal fluid, tears, saliva, cerebrospinal fluid, cell and/or bacterial culture supernatants, cervical swab, buccal swab, tissues, organs, and environmental materials.

17. The composition of claim 16, wherein the biological sample is blood.

18. The composition of claim 1, further comprising nucleic acids.

19. The composition of claim 18, wherein the nucleic acid is DNA or RNA, or a combination thereof.

20. The composition of claim 19, wherein the DNA is cell-free DNA.

21. The composition of claim 20, wherein the cell-free DNA is cell-free fetal DNA (cffDNA) or cell-free tumor DNA (cftDNA).

22. The composition of claim 1, further comprising a biological sample collection device.

23. The composition of claim 22, wherein the device is an evacuated collection container (e.g., a blood tube), a TAP blood collection device, a microcentrifuge tube, or a capillary blood collection tube.

24. A method comprising: contacting a biological sample comprising nucleic acids with a preservative, wherein the preservative comprises one or more anticoagulant agent, cell stabilizer agent, cryoprotective agent, hypertonic agent, or any combination thereof.

25. The method of claim 24, wherein the biological sample is contacted with the preservative in a collection device.

26. The method of claim 24, wherein the nucleic acids are DNA or RNA, or a combination thereof.

27. The method of claim 26, wherein the DNA is cell-free DNA.

28. The method of claim 27, wherein the cell-free DNA is cell-free fetal DNA (cffDNA) or cell-free tumor DNA (cftDNA).

29. A kit comprising: a sample collection device and a composition comprising one or more anticoagulant agents, one or more cell stabilizer agents, one or more cryoprotective agents, one or more hypertonic agents, or any combination thereof.

30. The kit of claim 29, wherein the composition is contained inside the sample collection device.

31. The kit of claim 29, wherein the composition in the kit is not contained in the sample collection device.
